(19) United States
(12) Patent Application Publication
(10) Pub. No.: US 2009/0004106 A1
(43) Pub. Date: Jan. 1, 2009

(54) Radioligands for the 5 -Ht1b Receptor

(76) Inventors: John Richard Heys, Litchfield, CT (US); Edward Pierson, Wilmington, DE (US); William Potts, Wilmington, DE (US)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(21) Appl. No.: 12/278,699

(22) PCT Filed: Feb. 14, 2007
(86) PCT No.: PCT/SE2007/000135

§ 371 Date: Aug. 7, 2008

Related U.S. Application Data
(60) Provisional application No. 60/773,302, filed on Feb. 14, 2006.

(51) Int. Cl. A61K 51/04; C07D 413/14

(52) U.S. Cl. 424/1.81; 544/121

(57) ABSTRACT

The present invention provides novel radioligands for the 5-HT_{1B} receptor of the formula I.

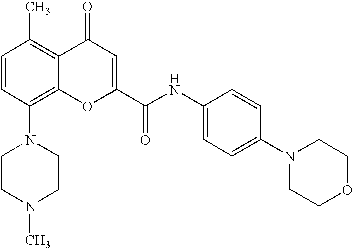

### FIELD OF THE INVENTION

The present invention provides for novel radioligands for the 5-HT_{1B} receptor.

### BACKGROUND OF THE INVENTION

Radioligands with high affinity and selectivity for specific receptors in the brain represent powerful tools in conducting a wide array of animal and human studies. For instance, certain radioligands in combination with Positron Emission Tomography (PET) or Single Photon Emission Computed Tomography (SPECT) can be used to measure receptor density, affinity and drug-induced receptor occupancy in animals including humans. The ability to measure receptor occupancy has proven particularly useful to correlate imaging with therapeutic effects and side effects of central nervous system drugs as well as for dose-finding studies in drug development.

Several more recent studies have demonstrated that noninvasive neuroreceptor imaging is also a useful approach to measure changes in the concentration of neurotransmitters. While PET and SPECT studies have commonly been used to study the dopaminergic system, they have yet to be extensively used to study the 5-HT system. This disparity is most likely due to a lack of suitable radioligands that are sensitive to changes in 5-HT concentrations. Initial animal studies with 5-HT_{1A} receptor antagonists such as [¹¹C]-WAY-100635 and [³H]-NAD299, have suggested some sensitivity to serotonin concentrations but have been difficult to replicate in humans.

While the therapeutic potential of drug action at the 5-HT_{1B} autoreceptor has been considered more recently, there is still need for the development of radioligands that would be suitable for in vivo studies of the 5-HT_{1B} receptor. The present invention meets this need by providing novel radioligands for the 5-HT_{1B} receptor.

### SUMMARY OF THE INVENTION

The present invention provides isotopically labeled compounds of formula I:

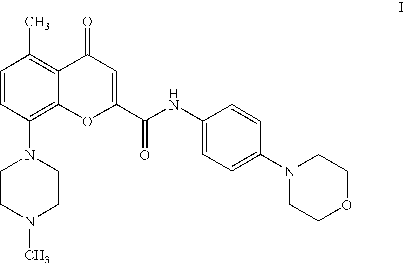

or a pharmaceutically acceptable salt thereof, wherein one or more atoms of the compound of formula I are replaced by an atom having an atomic mass or mass number different than the atomic mass or mass number usually found in nature for the atom.

### BRIEF DESCRIPTION OF THE DRAWINGS

FIG. 1 shows the regional distribution of an isotopically labeled compound in monkey brain.

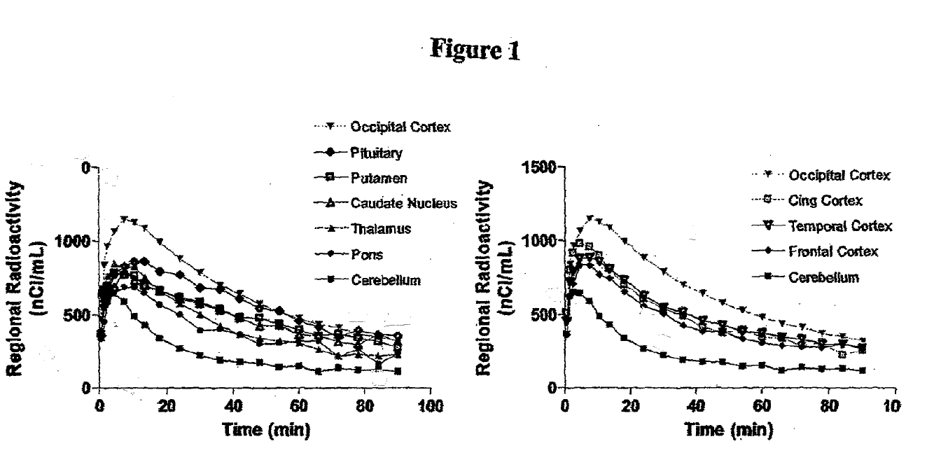

### DESCRIPTION OF EMBODIMENTS

Provided herein are isotopically labeled compounds of formula I:

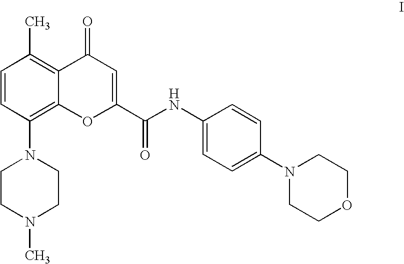

or a pharmaceutically acceptable salt thereof, wherein one or more atoms are, independently, replaced by isotopes of hydrogen, carbon, nitrogen, oxygen, having an atomic mass or mass number different than the atomic mass or mass number usually found in nature. Isotopic labels include, but are not limited to, ³H, ¹¹C, ¹⁴C, ¹³N, and ¹⁵O.

The present invention provides a compound according to formula I wherein the compound comprises at least one isotopic label selected from ³H, ¹¹C, ¹³N, and ¹⁵O.

The present invention provides a compound according to formula I wherein the compound comprises at least one isotopic label selected from ³H, or ¹¹C.

The present invention provides a compound according to formula I wherein the compound comprises ¹⁴C as the isotopic label.

The present invention provides a compound according to formula I wherein the compound is [³H] 8-(4-methylpiperazin-1-yl)-5-methylchrom-2-en-4-one-2-(4-morpholinophenyl)carboxamide).

The present invention provides a compound according to formula I wherein the compound is [¹¹C] 8-(4-methylpiperazin-1-yl)-5-methylchrom-2-en-4-one-2-(4-morpholinophenyl)carboxamide).

The present invention provides a compound according to formula II or a pharmaceutically acceptable salt thereof.

The present invention provides a pharmaceutical composition comprising the compound according to formula I and a pharmaceutically acceptable carrier.

The present invention provides the use of a compound according to formula I in positron emission tomography (PET) imaging of a 5-HT_{1B} receptor.

The present invention provides a method of making a pharmaceutical composition comprising mixing the compound according to formula I with a pharmaceutically acceptable carrier.

The present invention provides a method of identifying humans that would respond to a 5-HT_{1B} modulator by measuring the spatial distribution of [¹¹C] 8-(4-methylpiperazin-1-yl)-5-methylchrom-2-en-4-one-2-(4-morpholinophenyl)carboxamide) in the brain of such human.

The present invention provides a method of diagnosing depression in a human by measuring the spatial distribution of [¹¹C] 8-(4-methylpiperazin-1-yl)-5-methylchrom-2-en-4-one-2-(4-morpholinophenyl)carboxamide) in the brain of such human.

The present invention provides a method of monitoring depression in humans by measuring the spatial distribution of [¹¹C] 8-(4-methylpiperazin-1-yl)-5-methylchrom-2-en-4-one-2-(4-morpholinophenyl)carboxamide) in the brain of such human.

The present invention provides a method of identifying the dose of a 5-HT_{1B} modulator by measuring the spatial distribution of [¹¹C] 8-(4-methylpiperazin-1-yl)-5-methylchrom-2-en-4-one-2-(4-morpholinophenyl)carboxamide) in the brain of such human.

The present invention provides 8-(1-piperazinyl)-5-methylchrom-2-en-4-one-2-(4-morpholinophenyl)carboxamide or a pharmaceutically acceptable salt thereof as an intermediate in the making of formula I.

In addition, the present invention provides a non-invasive method for positron emission tomography (PET) imaging of one or more 5-HT_{1B} receptors in a mammal comprising labeling one or more 5-HT_{1B} receptors with an image generating amount of one or more of the isotopically labeled compounds of formula I and measuring spatial distribution of the compound in the mammal by PET.

Positron emission tomography (PET) is a technique for measuring the concentrations of positron-emitting isotopes within the tissues. These measurements are, typically, made using PET cameras outside of the living subjects. PET can be broken down into several steps including, but not limited to, synthesizing a compound to include a positron-emitting isotope; administering the isotopically labeled compound to a mammal; and imaging the distribution of the positron activity as a function of time by emission tomography. PET is described, for example, by Alavi et al. in Positron Emission Tomography, published by Alan R. Liss, Inc. in 1985.

Single-photon emission computed tomography (SPECT) acquires information on the concentration of isotopically labeled compounds introduced to a mammal's body. In general, SPECT requires isotopes that decay by electron capture and/or gamma emission. Subjects are injected with a radioactively labeled agent, typically at tracer doses. The nuclear decay results in the emission of a single gamma ray, which passes through the tissue and is measured externally with a SPECT camera. The uptake of radioactivity reconstructed by computers as a tomogram shows tissue distribution in cross-sectional images.

Depending on the intended mode of administration, the compounds of the present invention can be in pharmaceutical compositions in the form of solid, semi-solid or liquid dosage forms, such as, for example, tablets, suppositories, pills, capsules, powders, liquids, suspensions, lotions, creams, gels, or the like. The compositions can be in unit dosage form suitable for single administration of a precise dosage. In some embodiments the compound can be in a liquid form. In some embodiments, the compound is purified by HPLC, filtered through a sterile filter and administered intravenously to the individual.

The compositions can include an effective amount of the compound in combination with a pharmaceutically acceptable carrier and, in addition, may include other medicinal agents, pharmaceutical agents, carriers, adjuvants, diluents, etc. By pharmaceutically acceptable, it is meant a material that is not biologically or otherwise undesirable, i.e., the material may be administered to an individual along with the compound without causing any undesirable biological effects or interacting in a deleterious manner with any of the other components of the pharmaceutical composition in which it is contained.

In order that the invention disclosed herein may be more efficiently understood, examples are provided below. It should be understood that these examples are for illustrative purposes only and are not to be construed as limiting the invention in any manner.

### EXAMPLES

### Example 1

### Synthesis of Radioligand Precursor (8-(1-piperazinyl)-5-methylchrom-2-en-4-one-2-(4-morpholinophenyl)carboxamide)

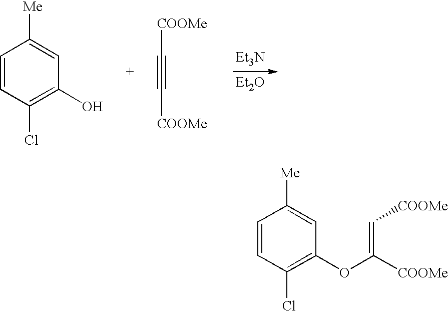

A 12.82 g (89.9 mmole) portion of 2-chloro-5-methylphenol can be dissolved in 75 mL of diethyl ether. A 10.9 g (107.9 mmole) portion of triethylamine is added dropwise with stirring, followed dropwise by 14.04 g (98.9 mmoles) of dimethyl acetylenedicarboxylate, producing a mild warming. The reaction mixture should be stirred overnight at room temperature, then transferred using rinses of ether, and a little THF to dissolve some residue, into a separatory funnel. The mixture should be washed two times with 200 mL of 1N NaOH, once with water, then twice with saturated NaCl, and finally dried over MgSO₄. Filtration and removal of solvent by rotary evaporation provides a pale yellow clear oil which is carried directly into the next reaction.

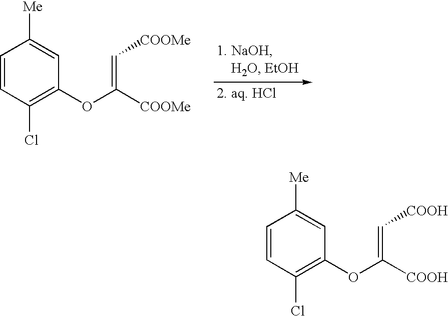

The oil from the preceding step can be taken up in 50 mL of EtOH and this solution can be added slowly with stirring to a solution of NaOH (14.4 g, 360 mmoles) in 50 mL of water at room temperature. The resulting clear amber-yellow solution can be heated to 80-90° C. for 2 hours then refluxed for 30 minutes. The reaction mixture should be cooled to room temperature, diluted with 200 mL of water, extracted twice with 150 mL of ether, then acidified by slow addition of conc. HCl with stirring. The resulting milky suspension can be extracted several times with ether and ethyl acetate, with addition of more conc. HCl to complete the protonation of acidic product. The ether and ethyl acetate extracts should be separately washed twice with saturated NaCl, dried (MgSO₄), filtered, and evaporated to give 5.44 g and 13.41 g, respectively, of O-(1,2-dicarboxyethenyl)-2-chloro-5-methylphenol as a pale yellow solid (82% over 2 steps).

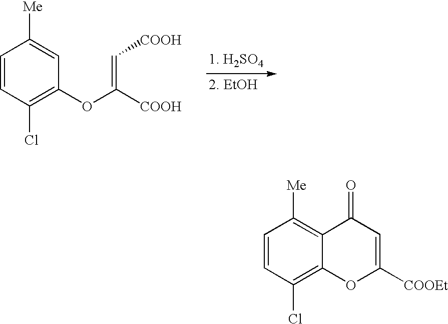

A 4.989 g (19.4 mmole nominally) portion of the O-(1,2-dicarboxyethenyl)-2-chloro-5-methylphenol can be dissolved, with warming, in 9 mL of concentrated sulfuric acid, then maintained at 80-90° C. for 10 minutes. The resulting dark viscous mixture should be added dropwise to a 55 mL portion of absolute ethanol maintained between about 80° C. and reflux temperature. After the addition is complete, the ethanol solution is refluxed for a few minutes, then reduced by about one-third in volume using a gentle stream of nitrogen. The solution should be then slowly cooled to −20° C. and allowed to stand overnight. The resulting crystalline solid can be collected by filtration then washed with cold 1:1 ethanol/water and dried in vacuo to give 2.568 g (50%) of 8-chloro-2-ethoxycarbonyl-5-methylchrom-2-en-4-one as silvery flakes.

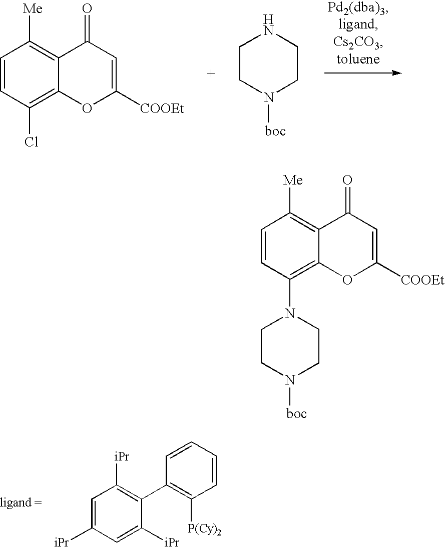

Under an inert atmosphere a 10 mL reaction vessel can be charged with 300 mg (1.12 mmole) of 8-chloro-2-ethoxycarbonyl-5-methylchrom-2-en-4-one, 312 mg (1.69 mmole) of 1-t-butyloxycarbonylpiperazine, 41 mg (0.042 mmole) of tris(dibenzylideneacetone)dipalladium(0), 53 mg (0.108 mmole) of dicyclohexyl[2-(2,4,6-triisopropylphenyl)phenyl]phosphine and 510 mg (1.57 mmole) of anhydrous cesium carbonate. An 8 mL portion of dry toluene is injected, and the reaction mixture is heated under an inert atmosphere with stirring in a 100-110° C. bath for approximately 16 hours. The reaction mixture is cooled to room temperature then partitioned between ethyl acetate and water. The organic layer should be separated, washed with saturated aqueous sodium chloride, dried over magnesium sulfate, filtered and evaporated to give 655 mg of orange oil with solid. Separation of the desired component on silica gel chromatography using methanol/dichloromethane provided 301 mg of orange oil with solid, from which 160 mg (34%) of 8-(4-t-butoxycarbonyl-1-piperazinyl)-2-ethoxycarbonyl-5-methylchrom-2-en-4-one can be isolated after trituration with ether.

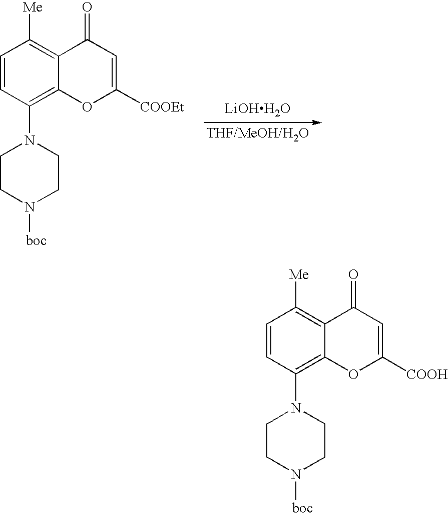

A 150 mg (0.373 mmole) portion of 8-(4-t-butoxycarbonyl-1-piperazinyl)-2-ethoxycarbonyl-5-methylchrom-2-en-4-one can be dissolved in 15 mL of 3:1:1 (v/v/v) THF/MeOH/H₂O. A 31 mg (0.75 mmol) portion of LiOH.H₂O is added and the mixture is stirred to homogeneity, then allowed to stand at room temperature for about 16 hours. Most of the organic solvent can be evaporated with a nitrogen stream, and the remaining solution is acidified to pH 3 by addition of 1N HCl. Extraction with ethyl acetate and washing of the extract with saturated brine, drying over magnesium sulfate, filtration and evaporation provides 141 mg (97%) of 8-(4-t-butoxycarbonyl-1-piperazinyl)-5-methylchrom-2-en-4-one-2-carboxylic acid as a yellow solid.

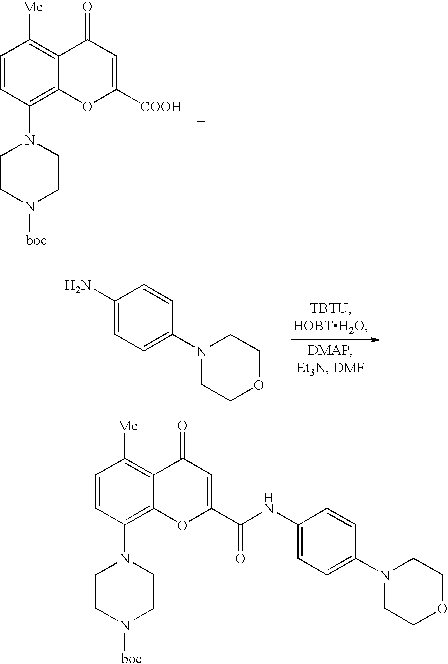

A 10 mL reaction vessel can be charged with 141 mg (0.363 mmole) of 8-(4-t-butoxycarbonyl-1-piperazinyl)-5-methylchrom-2-en-4-one-2-carboxylic acid, 65 mg (0.363 mmole) of 4-morpholinoaniline, 151 mg (0.472 mmole) of O-benzotriazol-1-yl-N,N,N′,N′-tetramethyluronium tetrafluoroborate, 64 mg (0.472 mmole) of 1-hydroxybenzotriazole hydrate and about 1 mg (about 0.009 mmole) of 4-dimethylaminopyridine. To this mixture is added in quick succession 4 mL of dry dimethylformamide and 101 μL (73 mg, 0.726 mmole) of triethylamine, and the mixture are stirred at room temperature for about 16 hours. Most of the solvent is evaporated in vacuo the residue is partitioned between chloroform and saturated sodium bicarbonate solution. The separated organic phase should be washed three times with water then saturated brine, dried over magnesium sulfate, filtered and evaporated to give 274 mg of dark amber-brown glass. Silica gel chromatography using methanol/dichloromethane provided 209 mg of dark amber-brown glass with crystals. Crystallization from ethanol/dichloromethane gives 111 mg (56%) of 8-(4-t-butoxycarbonyl-1-piperazinyl)-5-methylchrom-2-en-4-one-2-(4-morpholinophenyl)carboxamide. A further 69 mg is obtained in a second crop.

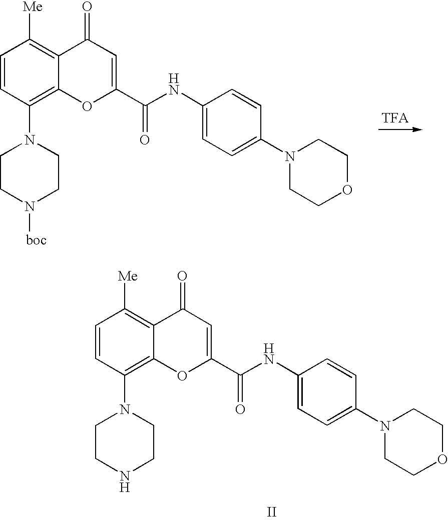

A 129 mg (0.235 mmole) portion of 8-(4-t-butoxycarbonyl-1-piperazinyl)-5-methylchrom-2-en-4-one-2-(4-morpholinophenyl)carboxamide can be dissolved in 6 mL of 1:1 (v/v) trifluoroacetic acid/dichloromethane and allowed to stand at room temperature for 30 minutes. The dark mixture is diluted with dichloromethane then basified by slow addition of saturated aqueous sodium bicarbonate. The organic layer should be separated, washed with saturated brine, dried over magnesium sulfate, filtered and evaporated to give 71 mg of yellow solid. Recrystallization from dichloromethane/hexane provides 61 mg (58%) of formula II 8-(1-piperazinyl)-5-methylchrom-2-en-4-one-2-(4-morpholinophenyl) carboxamide as fluffy yellow-green needles.

### Example 2

### Synthesis of Tritiated Radioligand [³H] 8-(4-methylpiperazin-1-yl)-5-methylchrom-2-en-4-one-2-(4-morpholinophenyl)carboxamide)

To 1.6 mg (3.57 umole) of precursor 8-piperazin-1-yl-5-methylchrom-2-en-4-one-2-(4-morpholinophenyl)carboxamide in 0.3 ml DMF is added 100 mCi of C³H₃I (in 100 μl DMF solution, Amersham) finally 100 μl DMF used as wash to give a total of 0.5 mL reaction volume. The reaction is sealed and placed in a 100-110° C. oil bath and heated for 50 minutes with stirring. The reaction mixture is allowed to cool and 6 mg of Boc anhydride added and heated sealed for 60 minutes. The volatiles can be removed and the product can be dissolved in acetonitrile and 0.1% TFA in water 50/50. The product is isolated on HPLC C18 Phenomex Luna column (10×50 cm; Gradient 20-60% acetonitrile (0.1% TFA) in 10 minutes (retention time=8.1 minutes). The major factions of separate isolation runs are combined; evaporated and redisolved in EtOH (6.3 mL) giving 26.8 mCi (4.25mCi/mL) at 80 Ci/mmole with 99% radiochemical purity.

### Example 3

### Synthesis of ¹¹C Radioligand: [¹¹C] 8-(4-methylpiperazin-1-yl)-5-methylchrom-2-en-4-one-2-(4-morpholinophenyl)carboxamide)

[¹¹C] 8-(4-methylpiperazin-1-yl)-5-methylchrom-2-en-4-one-2-(4-morpholinophenyl)carboxamide) can be prepared by methylation of the corresponding demethylated precursor 8-piperazin-1-yl-5-methylchrom-2-en-4-one-2-(4-morpholinophenyl)carboxamide) using [¹¹C]methyl triflate and purified by HPLC. The collected fraction from HPLC can be evaporated and the residue redissolved into 8 mL sterile physiological phosphate buffer (pH=7.4). After sterile filtration the formulated product solution will be sterile and free from pyrogens.

### Biological Testing

Cynomolgus monkey can be used to determine the suitability of [¹¹C] labeled raidoligands as a radioligand for PET-determination of 5HT^{1B}-receptor binding and occupancy. Anaesthesia can be induced and maintained by repeated intramuscular injections of a mixture of ketamine (3-4 mg/kg per h Ketalar, Parke-Davis) and xylazine hydrochloride (1-2 mg/kg per h Rompun® Vet., Bayer, Sweden) for the duration of each measurement. A fixation device can be used for the positioning of the monkey head during the PET-measurements. Body temperature can be controlled by Bair Hugger—Model 505 (Arizant Healthcare Inc, MN).

### PET Image Acquisition

Radioactivity in the brain can be measured with the Siemens ECAT Exact HR47 system that can be used in the three-dimensional model. The system covers an axial distance of 15 cm. The transaxial resolution of the reconstructed images is about 3.8 mm full width at half-maximum (FWHM) and the axial resolution 4.0 mm FWHM. Transmission scans can be acquired with three rotating ⁶⁸Ge—⁶⁸Ga sources and used to correct the emission scans for the attenuation of 511 keV photon rays through tissue and head support.

### PET Measurements in Monkeys

In each PET-measurement a sterile physiological phosphate buffer (pH=7.4) solution containing about 1.4 mCi (50 MBq) of [¹¹C] labeled compound can be injected as a bolus into a sural vein during 2 s with simultaneous start of PET-data acquisition. Radioactivity in brain can be measured continuously for 93 minutes according to a pre-programmed series of 15 frames starting immediately after IV injection of [¹¹C] labeled compound. The three initial frames can be 1 min each, the following three can be 3 minutes each and the remaining frames 6 minutes.

In each monkey a baseline measurement can be performed in the morning and a pretreatment measurement in the afternoon on the same day. In all pretreatment measurements the reference ligand can be injected i.v. 30 minutes prior to radioligand injection. All reference ligands can be injected in a slow bolus over a time frame of 3 to 5 minutes.

During all PET measurements venous blood samples (about 0.5 ml) can be taken at 4 time points (at about 4, 15, 30 and 45 minutes) and plasma radioactivity concentration determined in a well counter, cross-calibrated with the camera. The venous samples can also be used for determination of unchanged radioligand in plasma by HPLC.

### Regions of Interest (ROIs)

The ROIs can be drawn on the PET summation images representing radioactivity measured from 9 minutes after intravenous injection to the end of the measurement. The striatum, substantia nigra, pons, thalamus, hypothalamus, globus pallidus, frontal cortex, cerebellum and the whole brain contour is defined according to an atlas of a cryosected cynomolgus monkey head in situ. Radioactivity is calculated for the sequence of time frames, corrected for the radioactivity decay, and plotted versus time. For each region a time-activity curve can be generated and radioactivity expressed as nCi/ml.

To calculate the percentage of radioactive compound injected that is present in brain at time of maximal radioactivity the radioactivity concentration in the ROI for the whole brain is multiplied with the estimated brain volume of 65 mL for a cynomolgus monkey weighing 4 kg. The calculated value for total radioactivity in brain is then divided by the radioactivity injected and multiplied by 100 to obtain the percentage.

### Quantitative Analyses

The cerebellum is a region with negligible density of 5-HT_{1B}, and is used as a reference region for non-displaceable radioligand binding. The radioactivity in the cerebellar cortex is accordingly used as an approximate for free and non-specifically bound radioligand concentration in brain.

The time curve for specific radioactive ligand binding to 5-HT_{1B} in high-density regions is defined as the difference between the total radioactivity concentration in a ROI and the cerebellum. Time for peak equilibrium is defined as the moment when the curve for specific binding reaches its peak.

The ratio of binding in a ROI to the cerebellum is calculated in monkeys when peak equilibrium occurs. This ratio corresponds to the binding potential (BP) and can be viewed as an index for the density of available receptors. All calculations are based on the assumption that radioactivity in brain represents unchanged radioligand.

The compound in Example 3 rapidly entered the primate brain and binds specifically to the areas of interest with high enough specific to non-specific ratios (non specific defined as cerebellum) to be a useful primate PET ligand.

1. An isotopically labeled compound of formula I: 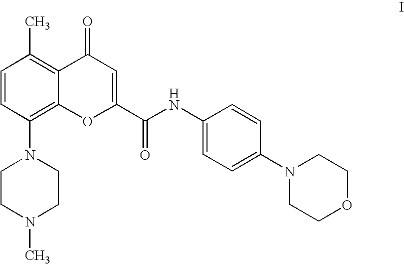
or a pharmaceutically acceptable salt thereof, wherein the compound comprises at least one isotopic label selected from ³H, ¹¹C, ¹³N, or ¹⁵O.

2. A compound according to claim 1 wherein the compound comprises at least one isotopic label selected from ³H, or ¹¹C.

3. A compound according to claim 1 wherein the compound comprises ¹⁴C as the isotopic label.

4. A compound according to claim 1 wherein the compound is [³H] 8-(4-methylpiperazin-1-yl)-5-methylchrom-2-en-4-one-2-(4-morpholinophenyl)carboxamide).

5. A compound according to claim 1 wherein the compound is [¹¹C] 8-(4-methylpiperazin-1-yl)-5-methylchrom-2-en-4-one-2-(4-morpholinophenyl)carboxamide).

6. A compound of formula II or a pharmaceutically acceptable salt thereof.

7. A pharmaceutical composition comprising the compound according to claim 1 and a pharmaceutically acceptable carrier.

8. The use of a compound according to claim 1 in positron emission tomography (PET) imaging of a 5-HT_{1B} receptor.

9. A method of making a pharmaceutical composition comprising mixing the compound according to claim 1 with a pharmaceutically acceptable carrier.

10. A method of identifying humans that would respond to a 5-HT_{1B} modulator by measuring the spatial distribution of [¹¹C] 8-(4-methylpiperazin-1-yl)-5-methylchrom-2-en-4-one-2-(4-morpholinophenyl)carboxamide) in the brain of such human.

11. A method of diagnosing depression in a human by measuring the spatial distribution of [¹¹C] 8-(4-methylpiperazin-1-yl)-5-methylchrom-2-en-4-one-2-(4-morpholinophenyl)carboxamide) in the brain of such human.

12. A method of monitoring depression in humans by measuring the spatial distribution of [¹¹C] 8-(4-methylpiperazin-1-yl)-5-methylchrom-2-en-4-one-2-(4-morpholinophenyl)carboxamide) in the brain of such human.

13. A method of identifying the dose of a 5-HT_{1B} modulator by measuring the spatial distribution of [¹¹C] 8-(4-methylpiperazin-1-yl)-5-methylchrom-2-en-4-one-2-(4-morpholinophenyl)carboxamide) in the brain of such human.
